(19) United States
(12) Patent Application Publication
(10) Pub. No.: US 2005/0021069 A1
(43) Pub. Date: Jan. 27, 2005

(54) Inflatable apparatus for accessing body cavity and methods of making

(76) Inventors: Gerald Feuer, Atlanta, GA (US); Michael Hogendijk, Mountain View, CA (US); Jeff Kraus, Los Gatos, CA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(21) Appl. No.: 10/898,587

(22) Filed: Jul. 23, 2004

Related U.S. Application Data
(63) Continuation-in-part of application No. 10/719,542, filed on Nov. 20, 2003.
(60) Provisional application No. 60/490,086, filed on Jul. 24, 2003.

(51) Int. Cl. A61M 29/00

(52) U.S. Cl. 606/192; 606/207

(57) ABSTRACT

Apparatus is provided for accessing a body cavity comprising a tubular member having a deflated insertion configuration and an inflated expanded configuration that facilitates viewing within the body cavity. The apparatus is formed by everting a length of material upon itself to form a tube having a single circumference seam at the distal end, and includes an inflation tube that is sufficiently rigid to assist in inserting the tubular member into a body cavity in the deflated insertion configuration. The tubular member further includes a pattern of staggered contact areas that configured so as to avoid the creation of longitudinal features that preferentially bend when loaded, thereby ensuring that tubular member provides a substantially circular central lumen.

### FIELD OF THE INVENTION

The present invention relates to methods and apparatus for accessing a body cavity, and more particularly, to methods and apparatus for gaining access to the female urogenitary tract.

### BACKGROUND OF THE INVENTION

Examination of the vagina and its associated anatomy is typically performed using a speculum, which provides access to the vagina by dilating the vaginal canal and then holding it in an expanded state. As currently used, a conventional speculum consists of a pair of metal jaws that are inserted into the vaginal canal and then actuated to expand the canal. For most patients, insertion and operation of the speculum is uncomfortable and may cause the patient to become tense, thus making a thorough examination difficult, if not impossible.

Speculums having inflatable exterior walls have been developed, such as described in U.S. Pat. No. 5,716,329 to Dieter. The speculum described in that patent includes a rigid interior wall and an inflatable exterior wall that may be inflated with fluid after insertion to alleviate discomfort associated with expansion of the vaginal canal. The device described in that patent, however, is fairly complicated and because it combines both reusable and disposable components, may not be commercially practicable.

In view of the low cost needed to have a commercially viable disposable speculum product, others have attempted to develop speculums that comprise inflatable sacs or ribs, such as described in International Patent Publication No. WO97/24975 and Dutch Patent No. 9100599. The products described in these publications and patents do not appear to possess sufficient expansile strength for practical use, however.

U.S. Pat. No. 5,743,852 to Johnson describes an inflatable speculum comprising an inflatable cone-like structure comprising inner and outer wall elements that are sealed together along their edges, and which further includes a grid of contact areas comprising a grid pattern. That patent describes an insertion rod disposed within the speculum to assist in insertion, and is coupled to an external sheath that is withdrawn through the central lumen of the device when the insertion rod is withdrawn. A cone-shaped structure that may be inserted within the inflated speculum once it is inflated to retain the speculum in the expanded state, and in addition, to provide support for a fiber-optic light or other instruments.

The foregoing Johnson patent appears to provide a number of advantages with respect to other inflatable speculum designs. However, the configuration of the insertion rod and sheath are expected to be problematic, in that the sheath is drawn from the distal (nearest the gynecologist) to the proximal edge (furthest within the patient) during removal, and may cause undue rubbing and discomfort. In addition, because the internal support structure disclosed in that patent does not extend to the proximal end of the speculum, it is possible for the forces applied by the patient's body to partially collapse the proximal end of the speculum. Finally, the use of sealed edges along the periphery of the inner and outer wall elements, especially at the proximal end of the speculum, may create a relative rigid structure capable of scraping the patient's cervix and causing patient discomfort.

U.S. Patent Publication US2003/1099737 to Deslauriers et al. describes an inflatable speculum having a plurality of longitudinally extending ribs arranged to delimit trapezoidal prisms within the volume of the speculum. As in the above-described WO publication and Dutch patent, the presence of the longitudinal ribs in the Deslauriers device is expected to preferentially distort to a central lumen of the speculum to a narrow elipse, rather than providing a substantially circular lumen.

In view of the aforementioned drawbacks of previously known devices, it would be desirable to provide methods and apparatus for accessing a body cavity that is small, easy to insert into the body cavity and comfortable once inserted and actuated within the body cavity.

It further would be desirable to provide methods and apparatus for accessing a body cavity that provides sufficient strength to expand the body cavity while using low-cost materials that permit the apparatus to be discarded after a single use.

It also would be desirable to provide apparatus for accessing a body cavity that provides sufficient radial strength to expand a body cavity in the vast majority of cases, but which may include a further optional component for use in special situations, e.g., in examining or treating obese patients.

It still further would be desirable to provide methods for manufacturing apparatus to access a body cavity that substantially eliminate the presence of longitudinal ribs or features that cause preferential bending of the device in the inflated state, and thereby ensure a substantially circular working lumen.

It also would be desirable to provide methods for manufacturing apparatus to access a body cavity that substantially eliminate the presence of welds or seals along the proximal peripheral edges of the device, thereby reducing the risk of patient discomfort.

### SUMMARY OF THE INVENTION

In view of the foregoing, it is an object of the present invention to provide apparatus for accessing a body cavity that is small, easy to insert into the body cavity and comfortable once inserted and actuated within the body cavity.

It is another object of this invention to provide methods and apparatus for accessing a body cavity that provide sufficient strength to expand the body cavity while using low-cost materials that permit the apparatus to be discarded after a single use.

It is another object of this invention to provide apparatus for accessing a body cavity that provides sufficient radial strength to expand a body cavity in the vast majority of cases, but which may include a further optional component for use special situations, e.g., in examining or treating obese patients.

It is a further object of the present invention to provide methods for manufacturing apparatus to access a body cavity that substantially eliminate the presence of longitudinal ribs or features that cause preferential bending of the device in the inflated state, thereby ensuring a substantially circular working lumen.

It is yet another object of this invention to provide methods for manufacturing apparatus to access a body cavity that substantially eliminate the presence of welds or seals along the proximal peripheral edges of the device, thereby reducing the risk of patient discomfort.

In accordance with the principles of the present invention, apparatus is provided for accessing a body cavity that comprises an inflatable body formed from a single sheet of material that is everted upon itself and sealed along its distal edge (nearest the physician), thereby eliminating the presence of a distal seal or weld zone and providing an atraumatic proximal end. The inflatable body is inserted into the body cavity in a deflated configuration and then inflated to an expanded configuration, thereby expanding the walls of the body cavity. The inflatable body includes at a plurality of contact points arranged in a substantially uniform pattern to permit substantially uniform pressure distribution within the inflatable body during expansion. In accordance with the principles of the present invention, the contact areas are arranged so as not to create substantially longitudinal features, but instead provides a substantially circular central lumen when the inflatable body is inflated under load.

The inflatable body is coupled to an inflation device, such as a bulb or pump, via a length of relatively stiff tubing that extends into and terminates within the inflatable body. The tubing is sufficiently rigid to permit the clinician to exert a force of the inflatable body, in the contracted delivery configuration, to drive the inflatable body into the patient's orifice. In addition, a retractable, pre-lubricated sheath may be disposed on the exterior of the inflatable body to assist in inserting the device into the patient's orifice.

Optionally, the apparatus includes an internal support member that may be inserted within the central lumen of the inflatable body after inflation. The support member preferably comprises an inexpensive plastic component that is mounted on a dilator, and then placed within the speculum to enhance the radial strength of the apparatus, and to prevent the proximal end of the inflatable body from collapsing. This optional support member may be particularly advantageous for use in obese patients.

In some embodiments, the inflatable body may include one or more pockets disposed within the central lumen of the inflatable body to permit a fiber-optic light or other instrument to be retained within the lumen. Alternatively, the inflatable body may include additional lengths of tubing that extend to a position near the proximal end of the apparatus to permit the evacuation of smoke generated during treatment of the organ, e.g., such as during leep-conization.

In accordance with other aspects of the present invention, the apparatus may be used to facilitate drug delivery within an organ or cavity. In some embodiments, the exterior surface of the inflatable body may be coated with one or more drugs that elute into the patient's tissue when the apparatus is disposed within the body or organ. In other embodiments, the inflatable body defines a receptacle that accepts a cylinder having one or more drug-filled chambers configured to fit within a lumen of the inflated inflatable body and provide a predetermined profile for release of the drugs.

In still further alternative embodiments, the apparatus includes a handle assembly that may be attached to the inflatable body to facilitate insertion of the inflatable body into the body cavity, or to re-orient the field of view accessible through the central lumen of the inflatable body.

Methods of manufacturing the apparatus of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a side view of the apparatus of the present invention in a deflated configuration;

FIGS. 2A and 2B are, respectively, cross-sectional views of the apparatus of FIG. 1 taken along view line 2-2 in the deflated and inflated states;

FIGS. 3A and 3B are, respectively, a side view and an end perspective view of the inflatable body of FIG. 1 in the inflated state;

FIG. 4 is a perspective view of a sheath for facilitating delivery of the inflatable body;

FIGS. 5A-5C are side views depicting an alternative design of the inflatable body of FIG. 1 including an optional side pocket and aspiration tube;

FIG. 6 is a side view, partly in section, of a support member and dilator for use with the apparatus of the present invention;

FIG. 7 is a perspective view of a preferred method of rolling the inflatable body to reduce patient discomfort during deployment;

FIG. 8 is a flow chart describing a preferred process for manufacturing the apparatus of FIG. 1;

FIG. 9 is a side view of the inflatable body of the present invention including a coating of a drug or other bioactive substance;

FIG. 10 is a cross-sectional view of an alternative embodiment of the inflatable body of the present invention suitable for delivering bioactive substances;

FIGS. 11A-11C are, respectively, side and perspective views of a handle assembly for use with the apparatus of the present invention, and a view depicting use of the handle assembly and inflatable body as a vaginal speculum;

FIGS. 12A-12C are, respectively, a side view of an alternative handle assembly for use with the apparatus of the present invention, and cross-sectional views of the handle assembly of FIG. 12A taken along lines 12B-12B and 12C-12C;

FIGS. 13A-13C are, respectively, a side view of a component of the handle assembly of FIG. 12A, a cross-sectional view of the component of FIG. 13A taken along line 13B-13B, and a side-sectional view of the component of FIG. 12A in use with the apparatus of FIG. 1;

FIG. 14 is a side-sectional view of an alternative embodiment of the device of FIG. 13C;

FIGS. 15A-15E are, respectively, side-sectional views of the device of FIGS. 12-13 within a patient's vaginal canal, a side-sectional view of the device of FIGS. 15A-15B with a cylinder disposed within the inflatable body, a cross-sectional view taken along line 15D-15D of FIG. 15C; and an end view of the cylinder of FIG. 15E;

FIGS. 16A-16E are, respectively, a side-sectional view of a patient's vaginal canal, side-sectional views of the device of FIG. 15 within the vaginal canal, and side-sectional views of the device of FIGS. 15B-15C with a cylinder within the central lumen;

FIG. 17 is a side-sectional view of the device of FIG. 15 being used to excise tissue from the cervix; and

FIG. 18 is a side-sectional view of the device of FIG. 15 being used to deliver radiation seeds within the vaginal cavity.

### DETAILED DESCRIPTION OF THE INVENTION

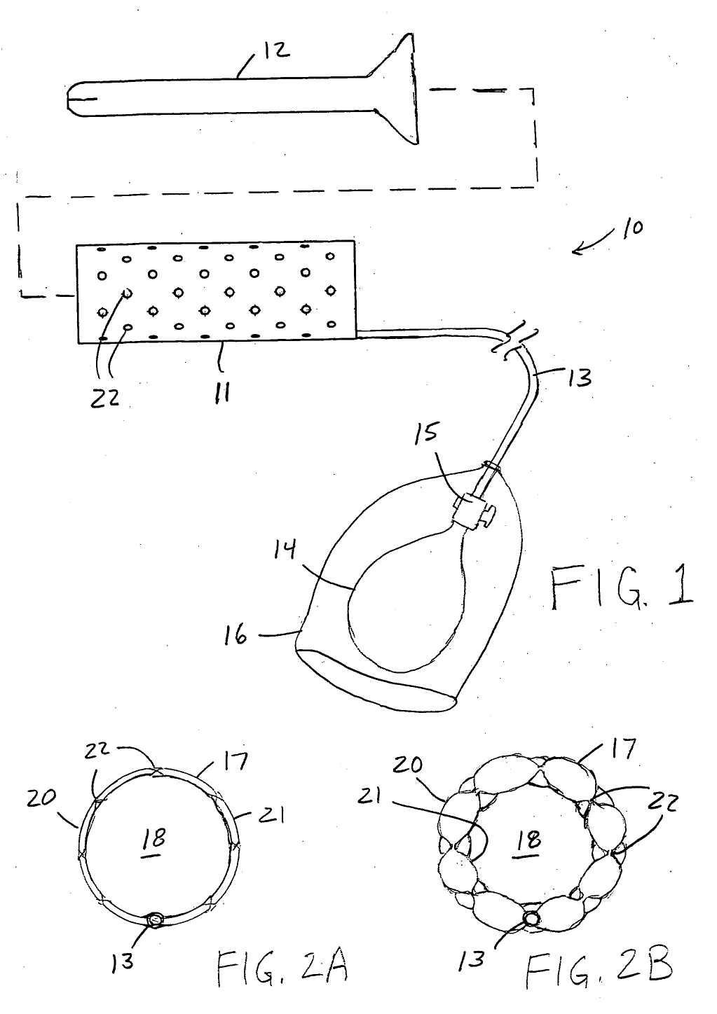

Referring to FIGS. 1-3, apparatus constructed the present invention provides low-cost single-use disposable apparatus for expanding a body cavity, such as the vaginal canal. Apparatus 10 comprises inflatable body 11, insertion sheath 12, inflation tube 13, inflator 14, valve 15 and shield 16. Inflatable body 11 transitions from a substantially flat tubular shape (FIG. 2A) to an expanded configuration (FIG. 2B) when inflated using inflator 14, illustratively a bulb.

In the expanded configuration, inflatable body 11 forms annular main body portion 17 defining central lumen 18 that provides the physician with access to the interior of the body organ or lumen. In accordance with one aspect of the present invention, inflation tube 13 is bendable but is otherwise relatively stiff, so that force applied to the inflation tube may be used to push the inflatable body into a patient's orifice. Inflation tube 13 communicates with the interior of main body portion 17 to permit inflatable body 11 to be inflated and deflated.

Valve 15 preferably is a one-way valve that retains pressure within main body portion 17, but does not require that bulb 14 remain pressurized. Valve 15 may be selectively actuated to deflate main body portion 17. Bulb 14 and valve 15 preferably are coupled to inflation tube via a conventional luer fitting, so that these items may be uncoupled from inflatable body 11 and inflation tube 13 for subsequent reuse. Bulb 14 and valve 15 preferably are disposed within shield 16, e.g., a plastic bag, to prevent contamination with the patient's body fluids.

Insertion sheath 12 comprises a light-weight plastic sheath that restrains in inflatable body 11 in a contracted position to facilitate insertion in the patient's organ or lumen. Sheath 12 includes a split bullet-nosed atraumatic shape that assists in insertion of the device, and is retracted distally over inflation tube 13 during deployment of the inflatable body.

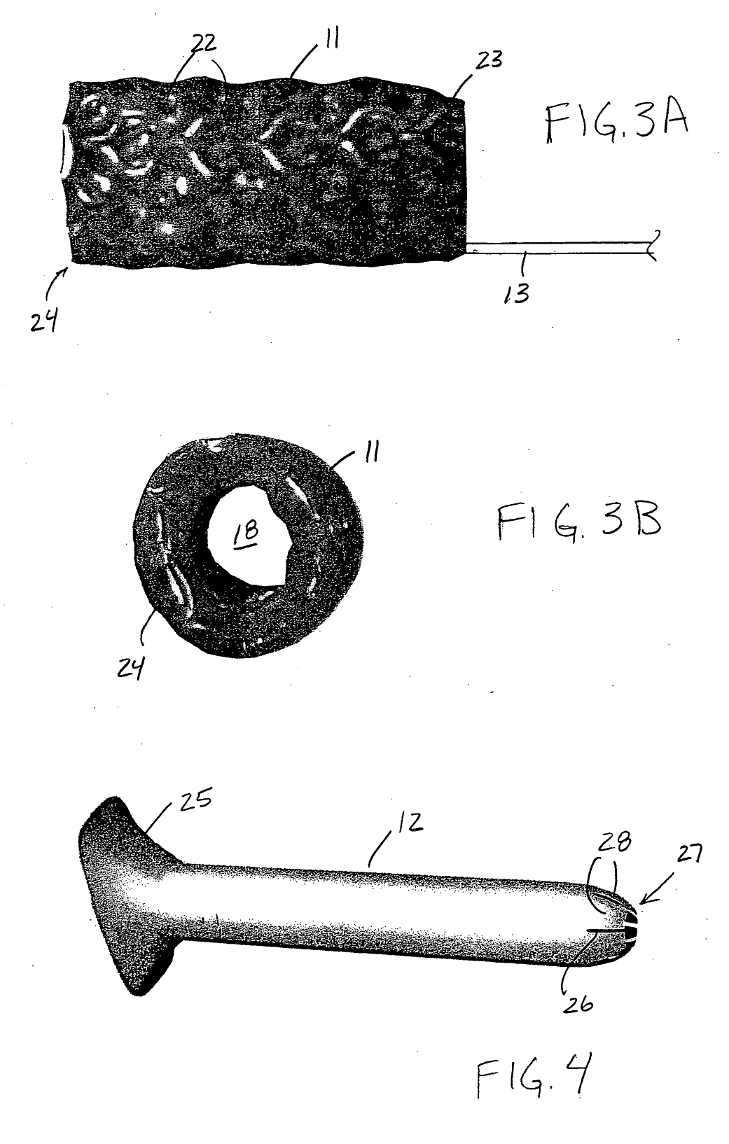

As depicted in FIGS. 2 and 3, inflatable body 11 preferably comprises a polymeric, latex-free material and is formed so that exterior wall 20 is joined to interior wall 21 at plurality of pillow-like quilted contact areas 22. Preferably, contact areas 22 are arranged in a uniform pattern to allow for substantially uniform pressure distribution within the inflatable body 11 during expansion. In a preferred embodiment, 16 rows of contact areas are provided around the circumference of the inflatable body and axially offset.

In accordance with the principles of the present invention, distributing the rows of contact areas 22 in an axially offset or staggered arrangement avoids the creation of longitudinal features on the inflatable body. Such features, which are present in the previously known devices, lead to preferential bending of the device under load, and permit the central lumen to become distorted into a narrow elipse. The offset grid pattern illustrated in FIG. 1, however, enhances the radial stiffness of the inflatable body in the expanded configuration, and ensures that central lumen 18 remains substantially circular in the inflated state, even under load.

Referring to FIG. 3, inflatable body 11 preferably comprises a single piece of material that is everted onto itself to form a double-layer tubular annulus that is approximately half as long as the original piece of material. In this manner, seam or weld 23 is formed only along one end of the inflatable body, as indicated in FIG. 3A, preferably at the distal end of the inflatable body (nearest the physician). This avoids the presence of a seam or weld at proximal end 24 of the inflatable body, and instead provides a soft, pillow-like atraumatic proximal end that reduces the risk of scraping or injuring tissue within the organ or lumen, as shown in FIG. 3B.

With respect to FIG. 4, insertion sheath 12 comprises a soft polymer tube, such as heat-shrinkable tubing, that retains inflatable body 11 in a contracted insertion configuration. Sheath 12 includes distal flange 25 to provide the clinician with a grip to grasp and withdraw the sheath distally. Sheath 12 also includes slots 26 in bullet-shaped nose 27 that permits leaves 28 between slots 26 to open outward during retraction of the sheath.

Insertion sheath 12 preferably is lubricated with a biocompatible lubricant and then inserted into a patient's body cavity, e.g., the vagina. In accordance with one aspect of the present invention, inflation tube 13 is sufficiently rigid that it permits the clinician to hold the inflatable body stationary within the body cavity with one hand, while retracting the insertion sheath from the inflatable body in a distal direction with the other hand. The insertion sheath is then removed over the luer at the distal end of inflation tube 13, and valve 15, bulb 14 and shield 16 then are coupled to the luer to permit the inflatable body to be inflated.

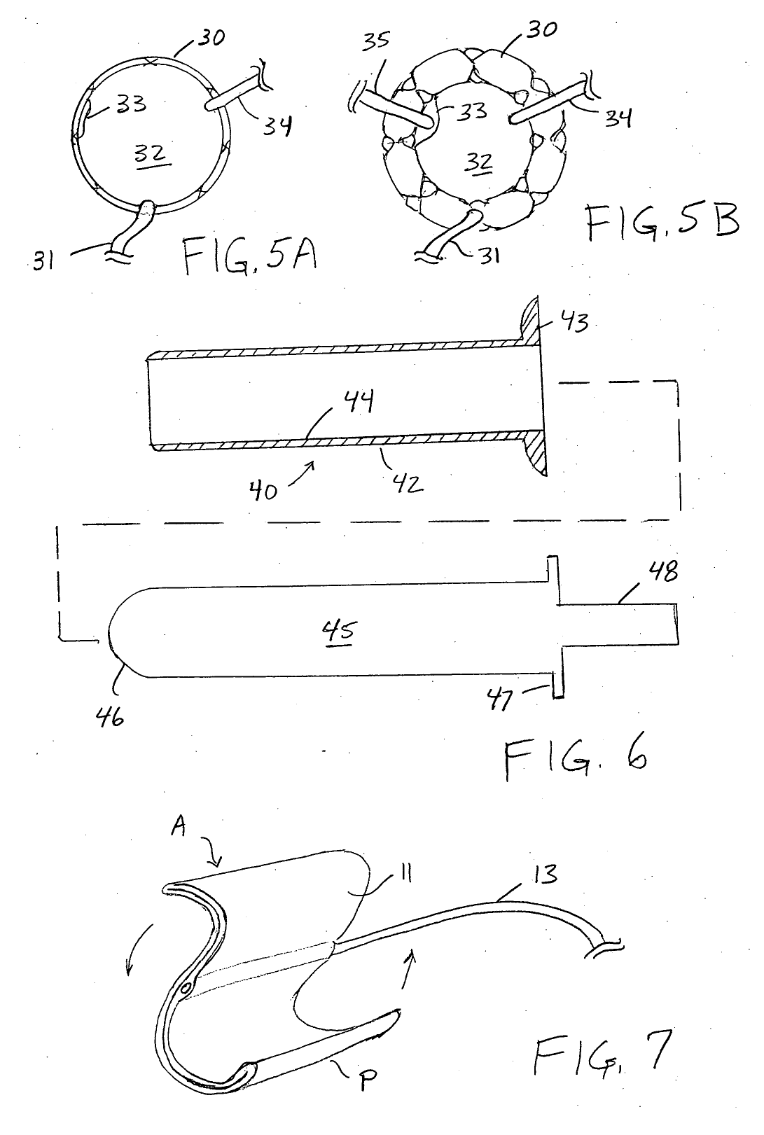

Referring now to FIGS. 5A and 5B, an alternative embodiment of the inflatable body of the present invention, suitable for use in colposcopy, leep-conization or other procedures, is described. Colposcopy is a procedure that looks at the cervix and vagina using glasses or other optical devices and generally requires vaginal illumination. Leep-conization is a procedure wherein an electrically-powered snare is used to remove tissue from an interior surface of the patient's cavity or organ, and can lead to the generation of smoke that must be evacuated to provide the physician with a clear field of view.

In FIGS. 5A and 5B, inflatable body 30 includes inflation tube 31, central lumen 32 having pocket or channel 33 and evacuation tube 34 fitted along its length. Channel 33 may be used to secure tool 35, such as fiber optic light source or other instrument, in position within central lumen 32. Evacuation tube 34 preferably extends the length of inflatable body 30 and includes a distal termination that permits tube 34 to be coupled to a suitable vacuum source to evacuate smoke or gases from within the body cavity during a procedure. Advantageously, channel 33 and evacuation tube 34 free up the physician's hands for other tasks. Alternatively, a light source to illuminate the body cavity may be substituted for evacuation tube 34 to facilitate procedures that require vaginal illumination, and channel 33 used to retain another instrument.

Referring to FIG. 6, optional support member 40 of the present invention is described. During initial testing it has been observed that in a certain segment of the population, especially obese women, the inflatable body of the present invention may not provide sufficient radial strength to provide a clear field of view through the central lumen. In FIG. 6, support member 41 comprises rigid disposable plastic tube 42 having distal flange 43 and central lumen 44. Tube 42 is dimensioned to accept dilator 45 within central lumen 44. Dilator includes smooth proximal end 46, flange 47 and handle 48. Flange 47 is configured to abut against flange 43, so that force exerted on handle 48 urges dilator 45 and tube 42 within the central lumen of the inflatable body of the apparatus of FIG. 1.

An illustrative use of the apparatus of FIGS. 1 and 6 as a vaginal speculum for obese women is now described. First, the inflatable body, disposed within insertion sheath 12 is inserted into the vagina. Inflation tube 13 then is held stationary while the insertion tube is withdrawn distally. Bulb 14, valve 15 and shield 16 then are attached to the luer termination of inflation tube 13 and the inflatable body is inflated. Dilator 45, with tube 42 disposed thereon, is then inserted into the central lumen of the inflatable body and driven forward by applying a proximally-directed force to handle 48. Once dilator 45 and tube 42 are fully inserted, dilator 45 is withdrawn, leaving tube 42 in position within the central lumen of the inflatable body. Advantageously, because tube 42 preferably extends to the proximal extremity of the inflatable body, it provides a clear field of view all the way to the patient's cervix.

With respect to FIG. 7, a preferred method of rolling the inflatable body of FIG. 1 to minimize discomfort during deployment is described. The present inventors have observed that in conventional jaw-type specula, the forces applied by the jaws are primarily in the anterior and posterior directions. This is believed to be so because lateral forces applied to the vagina are believed to cause discomfort. Accordingly, in accordance with one aspect of the present invention, the inflatable body is first flattened and then rolled in an S-shaped configuration having an anterior directed wing A and a posterior-directed wing P, as depicted in FIG. 7. When rolled in this manner, the forces applied to the vaginal walls during deployment of the inflatable body are primarily in the anterior and posterior directions, thereby reducing patient discomfort during inflation of the device.

Referring now to FIG. 8, a method of making the apparatus of FIGS. 1-3 is described. At step 50, a rectangular piece of plastic sheet, such as 8 mil urethane, is cut to a desired size. For example, if the inflatable body is to be made having a nominal length of 12 cm and expanded diameter of 3.8 cm, the corresponding sheet size may be 15 cm×23 cm. At step 51, the sheet is formed into a cylinder, and a longitudinal seam is formed. At step 52, a length of inflation tube is affixed to exterior of cylinder for a distal one-half of length of cylinder. At step 53, the proximal one-half length of cylinder is everted over distal one-half of cylinder to form double-walled annular tube.

At step 54 a seal or weld is formed at the distal end of double-walled annular tube, thereby forming closed tube. As described hereinabove, having the weld only at the distal end of the inflatable body provides a smooth, atraumatic proximal end to the inflatable body. At step 55 a pattern of contact areas are formed along length and circumference of double-walled annular tube to form the inflatable body. As also described above, the contact areas are axially offset or staggered, so that when the inflatable body is inflated, no predominantly longitudinal features form that preferentially permit bending or partial collapse of the tube, as in previously known designs.

At step 56, shield 16 may be applied to the inflation tube, and at step 57, the luer termination may be applied to the distal end of the inflation tube. Alternatively, the luer termination may be applied, and the shield separately applied at a later time, e.g., after the insertion tube has been removed. Once the inflatable member is completed, at step 57, it may be rolled into an S-shaped configuration, as described above with respect to FIG. 7, and inserted into an insertion sheath at step 58. In subsequent steps, the insertion tube may be heated to cause it to shrink down on the inflatable body, and the device may then be packaged and sterilized.

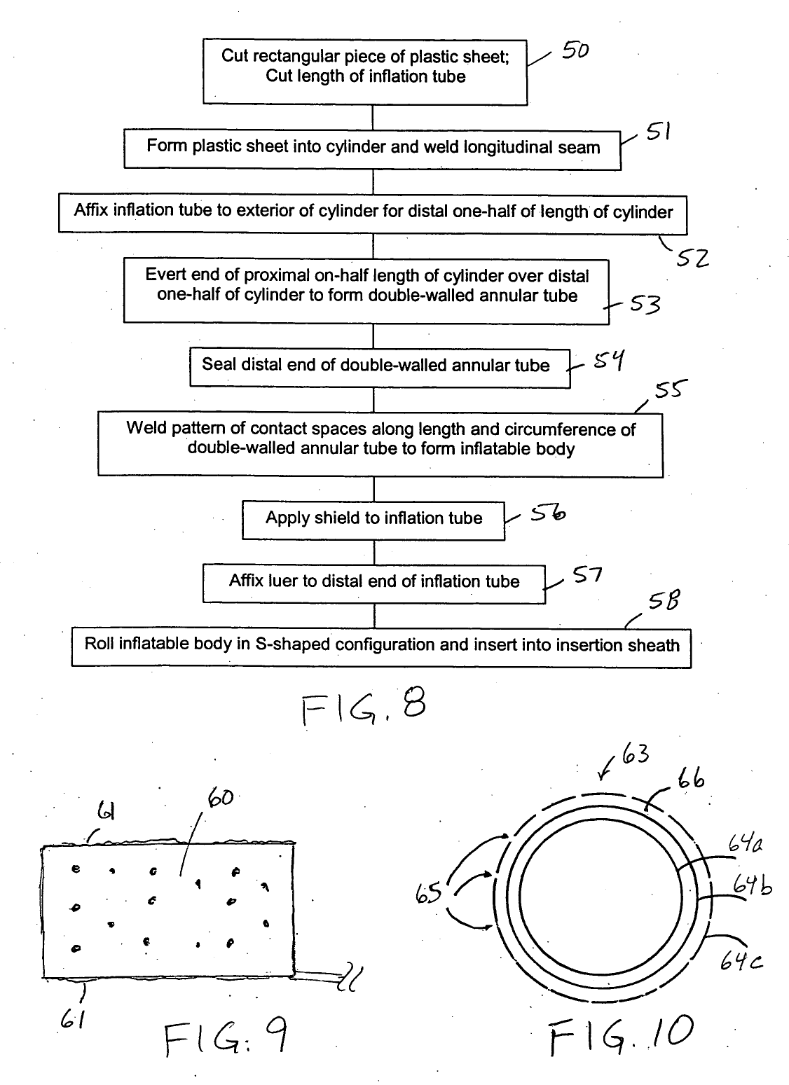

With respect to FIG. 9, an alternative embodiment of the inflatable body of the present invention is described. Inflatable body 60 is similar in construction to the embodiment of FIG. 1 described above, but in addition includes coating 61 containing a drug, e.g., an antibiotic, for topical distribution within the body cavity or lumen. Alternatively, coating 61 may comprise a gene vector or protein coating. By providing the coating on the exterior wall, the drug, gene vector or protein may be delivered directly to the vaginal wall during examination and treatment. By way of example, coating 61 may contain Novocain, contraceptives, fertilization preparations, coagulants and various genes and proteins. Depending upon the pharmicokinetics of various drugs, genes and proteins and how they are absorbed in the vagina, coating 61 may contain more than one drug to be delivered into the vagina.

To facilitate delivery of the drug, gene or protein, features or patterns may be provided on the exterior wall. Alternatively, coating 61 may be lubricious and become slippery when exposed to water, thus reducing friction encountered during insertion of the device. As a further alternative, the apparatus may be pre-soaked in warm water prior to insertion to reduce patient discomfort, as the inflatable body is expected to retain some of the heat from the warm water.

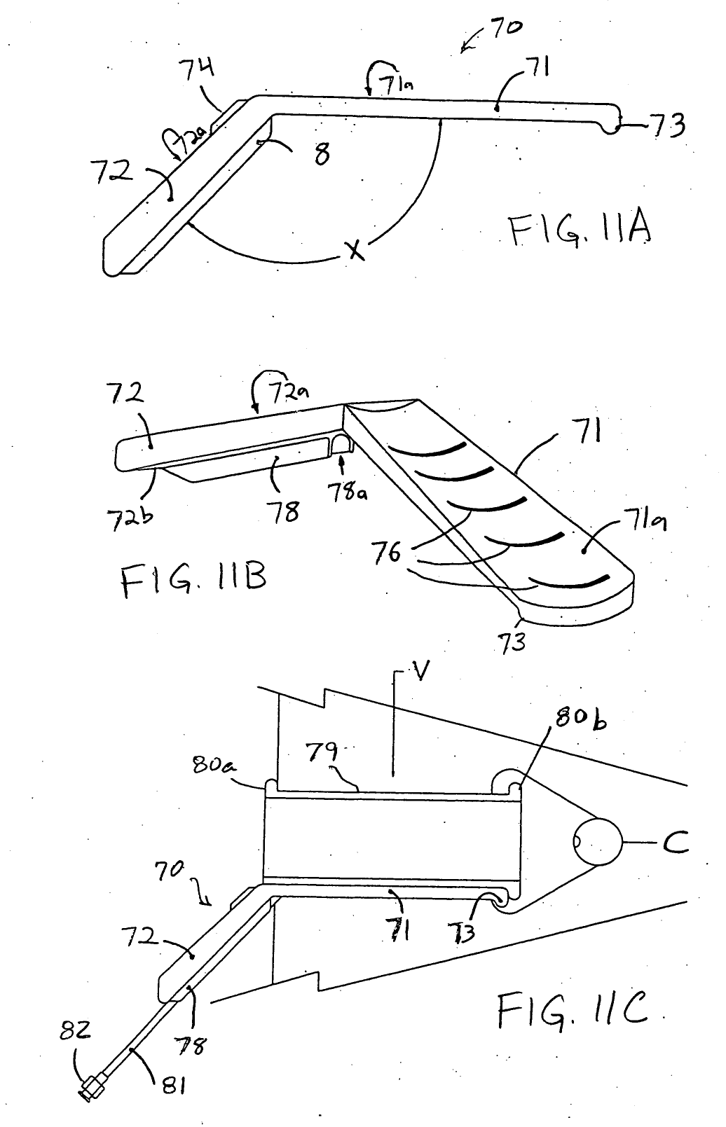

In a further alternative embodiment depicted in FIG. 10, inflatable body 63 comprises interior layer 64a, middle layer 64b and exterior layer 64c. Interior layer 64a and middle layer 64b correspond to interior wall 21 and exterior wall 20 in the embodiment of FIG. 2A, while exterior layer 64c includes plurality of micro-perforations 65. The annulus between interior layer 64a and middle layer 64b is filled with gas or fluid to expand inflatable body 63, while the annulus between middle layer 64b and exterior layer 64c forms pocket 66, which may be filled with drugs, proteins or gene-vectors in a liquid or gel form. When the inflatable body is expanded inside a patient's body cavity, the drugs, proteins or gene-vectors within pocket 66 are forced through micro-perforations 65 and delivered to the wall of the body cavity.

With respect to the embodiments of FIGS. 9 and 10, coating 61 or pocket 66 may include medications for treating yeast infections, such as Terazol, Diflucan, Monistat and Gynazole. Alternatively, coating 61 or pocket 66 may include medications for treating bacterial infections, such as flagy and cleocin.

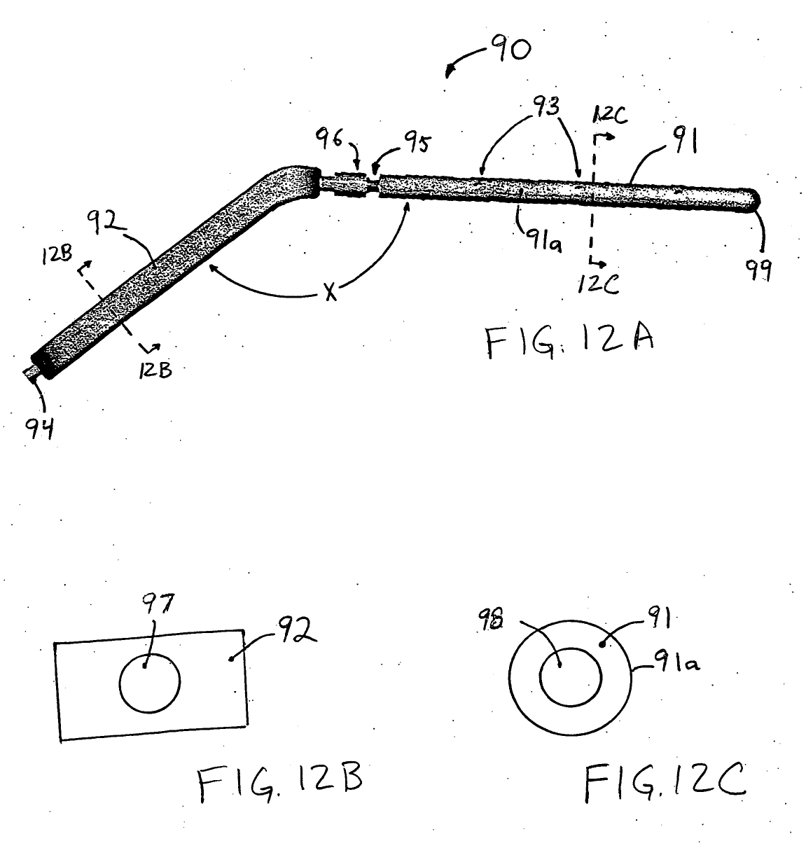

FIGS. 11A-11C depict handle assembly 70 configured for use with inflatable body 11 described with respect to FIGS. 1-3. Handle assembly 70 includes intravaginal tongue portion 71 and gripping portion 72 for holding and manipulating the handle assembly. Tongue portion 71 includes anterior surface 71a, which is preferably concave to match the exterior contour of inflatable body 11. Handle assembly 70 facilitates insertion of the deflated device and manipulation of the internal anatomy of the vagina. Tongue portion 71 preferably includes lip 73 configured to engage the patient's vaginal canal such that the cervix can be manipulated and viewed.

Referring to FIG. 11A, tongue portion 71 is disposed at an angle X relative to gripping portion 72, thereby permitting a user to communicate substantial leverage to tongue portion 71 when holding gripping portion 72. Preferably, angle X is greater than 90 degrees, more preferably between 120 and 160 degrees. This angle between the gripping and tongue portions facilitates insertion of the device and lessens the need to reposition the patient. Advantageously, this allows the patient to sit or lie in a more comfortable position during most examinations.

In some embodiments gripping portion 72 includes thumb rest 74 designed to promote comfortable gripping of gripping portion 72 during a procedure. Thumb rest 74 is disposed generally on an anterior surface 72a of gripping portion 72 near the junction of the gripping and tongue portions. Thumb rest 74 preferably comprises a material, e.g., rubber, that permits the handle assembly to be gripped securely while enhancing the tactile sensation of the user.

As shown in FIG. 11B, the anterior surface of tongue portion 72 optionally may include measurement indicia comprising radiopaque markings 76 that are visible under fluoroscopic examination. Radiopaque markings 76 permit measurements to be taken with respect to surrounding objects such as organs, tumors, tissue and bones. By way of example, radiopaque markings 76 may be used to determine the depth or location of a tumor. Posterior surface 72b of gripping portion 72 includes guide 78 including substantially U-shaped channel 78a for securely supporting inflation lumen 13 during a procedure. Guide 78 may comprise a flexible material that allows the inflation lumen to be force-fit or snap-fit within U-shaped channel 78a.

In FIG. 11C, inflatable body 79, including optional cuffs 80a and 80b, is attached to the anterior surface of tongue portion 71, so that the inflatable body deploys outwardly from the anterior surface. Inflatable body 79 preferably is attached to tongue portion 71 using a suitable adhesive, ultrasonic welding or heat welding. Alternatively, the inflatable body may be attached to the tongue portion using a quick connector. In addition, inflation lumen 81 may be force-fit within flexible guide 78 and preferably includes connector 82, such as a conventional luer-type connector for attachment to a suitable inflator, e.g., a pump or bulb.

In FIG. 11C, inflatable body 79 and handle assembly 70 are positioned within vaginal canal V of a patient. Lip 73 and cuff 80b preferably are configured to engage the end of vaginal canal V such that the patient's cervix C can be manipulated and viewed. Forces applied to the gripping portion by the physician produce resultant forces applied to the inside of the terminus of the vaginal canal and subsequent anatomy. Manipulation of the gripping portion therefore causes the cervix to present, thereby allowing the physician to view the junction between the cervix and vaginal canal at any desired angle.

FIGS. 12A-12C depict alternative handle assembly 90 adapted for use with inflatable body 11 of FIGS. 1-3. Handle assembly 90 is a modular assembly including intravaginal tongue portion 91 and detachable gripping portion 92. Preferably the tongue and gripping portions are releasably connected using conventional luer-type connectors 95 and 96, per se known in the art. More particularly, the end of tongue portion 91 includes male luer component 95 adapted to mate with a corresponding female luer component 96 disposed on gripping portion 92. These luer components preferably incorporate a standard twist-lock feature for engagement and disengagement. Alternatively, the tongue and gripping portions may be releasably connected by way of friction-fit, force-fit or snap-fit. Preferably, the tongue and gripping portions are easy to attach and detach, but will not inadvertently detach during use.

As depicted in FIG. 12A, tongue portion 91 preferably includes convex anterior surface 91a, which is configured to match the interior contour of inflatable body 11. In the illustrated embodiment, tongue portion 91 is substantially cylindrical and anterior surface 91a includes one or more inflation holes 93 for expanding inflatable body 11. Gripping portion 92 includes a female luer component 94 configured to mate with a corresponding male luer component of an inflator.

FIG. 12B is a cross-sectional view of the gripping portion of FIG. 12A taken along line 12B-12B and FIG. 12C is a cross-sectional view of the tongue portion of FIG. 12A taken along line 12C-12C. Gripping portion 92 includes lumen 97 that runs the length of the gripping portion and is in communication with the inflation device via connector 91. Although the cross-section of the gripping portion is depicted as rectangular, it also may be other shapes including, but not limited to, square, circular, triangular and elliptical, without departing from the scope of the present invention.

With respect to FIG. 12C, tongue portion 91 also includes lumen 98 that runs the length of the tongue portion and is in communication with both lumen 97 and inflation holes 93. Although the cross-section of the gripping portion is depicted as circular, it also may be other shapes including, but not limited to, square, rectangular, triangular and elliptical, without departing from the scope of the present invention.

Referring again to FIG. 12A, tongue portion 91 is disposed at an angle X relative to gripping portion 92, thereby permitting a user to communicate substantial leverage to tongue portion 91 when holding gripping portion 92. Preferably, angle X is greater than 90 degrees, more preferably between 120 and 160 degrees. Such an angle between the gripping and tongue portions facilitates insertion of the device and lessens the need to reposition the patient. As noted hereinabove, this advantageously allows the patient to sit or lie in a more comfortable position during most examinations.

Similar to the previous embodiment, handle assembly 90 facilitates insertion of the deflated device and manipulation of the internal anatomy of the vagina. Tongue portion 91 includes tip 99 configured to engage the end of a patient's vaginal canal so that the cervix may be manipulated and viewed. The tongue portion provides for the manipulation of the patient's cervix by engaging the junction of the vaginal canal. Both the tongue and gripping portions preferably comprise hollow plastic pieces fabricated, e.g., using an injection molding process. Alternatively, the tongue and gripping portions may comprise other materials, such as metal or wood.

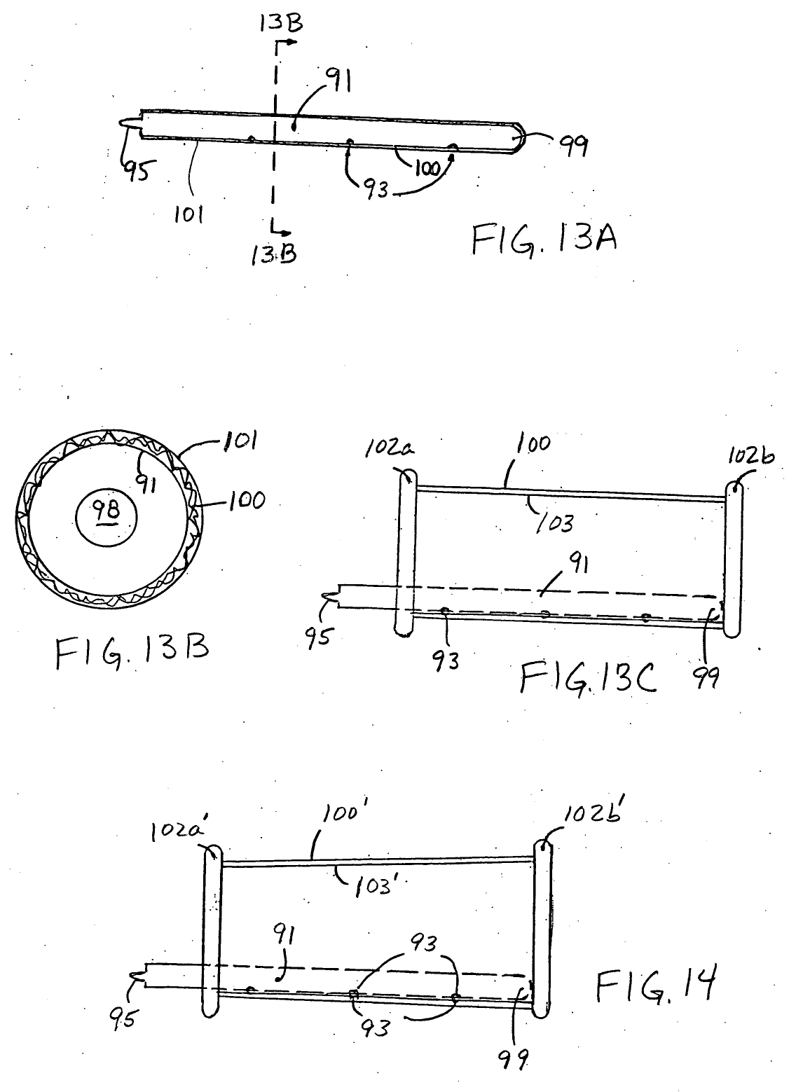

Referring to FIGS. 13A-13C, prior to insertion, the unexpanded inflatable body is attached to tongue portion 91 so that inflation holes 93 in tongue portion 91 are aligned with corresponding inflation holes in the interior wall of the inflatable body 100. Inflatable body 100 is similar in construction to the inflatable body of FIGS. 1-3, and includes interior cuffs 102a and 102b and interior wall 103. Preferably, the tongue portion is disposed within the relatively soft inflatable body 100, thereby providing maximum comfort for the patient. Inflatable body 100 may be attached to tongue portion 91 using a suitable adhesive, ultrasonic welding or heat welding.

Sheath 101 is employed to facilitate insertion of the tongue portion and inflatable body. Sheath 101 is adapted to hold the inflatable body against the tongue portion during insertion. Additionally, sheath 101 is adapted to split open during expansion of inflatable body 100. Sheath 101 may include a series of perforations to facilitate splitting open during inflation. Sheath 101 may be removed by the physician after the procedure or, alternatively, may be configured to dissolve within the vaginal canal.

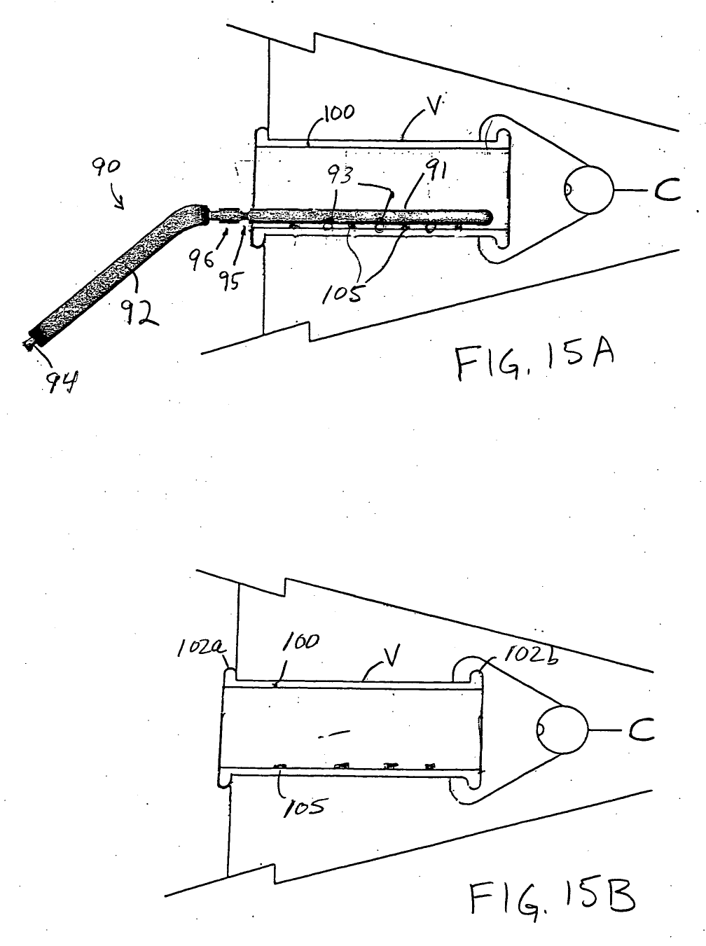

In another alternative embodiment depicted in FIG. 14, inflatable body 100′ is tapered so that, in the expanded configuration, the proximal end is larger in diameter than the distal end. In addition, cuff 102b′ is larger in diameter than cuff 102a′. This configuration assists in keeping the expanded inflatable body 100′ in place within a patient's vaginal canal.

Preferably, a patient will be given the option of inserting the speculum by herself. Advantageously, it is easier for a patient to insert the tongue portion once the handle portion has been removed. As shown in FIG. 13A, tongue portion 91 resembles a tampon having an elongated cylindrical surface and bullet-shaped distal tip 99. After insertion, the gripping portion optionally may be attached by way of the luer-type connectors 95 and 96. In many instances, the cervix may be visualized without any manipulation of the handle assembly after insertion into the vagina. In these cases, there is no need to attach the gripping portion, as the female luer connector may be attached directly to the inflator. However, in cases where the cervix cannot be visualized, the gripping portion may be attached to the tongue portion to provide the appropriate leverage for manipulating tongue portion 91.

Tongue portion 91 is designed to be thick enough to be usable as a lever without breaking, yet thin enough to provide comfort for the patient. Additionally, tongue portion 91 preferably is available in varying sizes for the treatment of different patients. Preferably, the tongue portion has a diameter of approximately 10 mm to 15 mm. Of course, as would be understood to those of ordinary skill in the art, the tongue portion may have a diameter other than 10 mm to 15 mm without departing from the scope of the present invention.

Inflatable body of the present invention preferably comes in multiple sizes, including a small size designed for young women and atrophic postmenopausal women, a medium size designed for “normal” women, and a large size designed for obese women. The small size preferably has a length of about 10 cm and a diameter of about 2.5 cm. The medium size preferably has a length of about 12 cm and a diameter of about 4.0 cm. The large size preferably has a length of about 18 cm and a diameter of about 6 cm.

The vagina and rectum are natural organs that are capable of effectively absorbing certain drugs into the venous system. Currently, there exist hormone devices that are placed into the vagina to allow continuous administration of one or more drugs. However, such devices are difficult and often painful to insert. Additionally, several devices of varying sizes may have to be installed before finding a good fit, and some patients may be between sizes such that a good fit is unattainable.

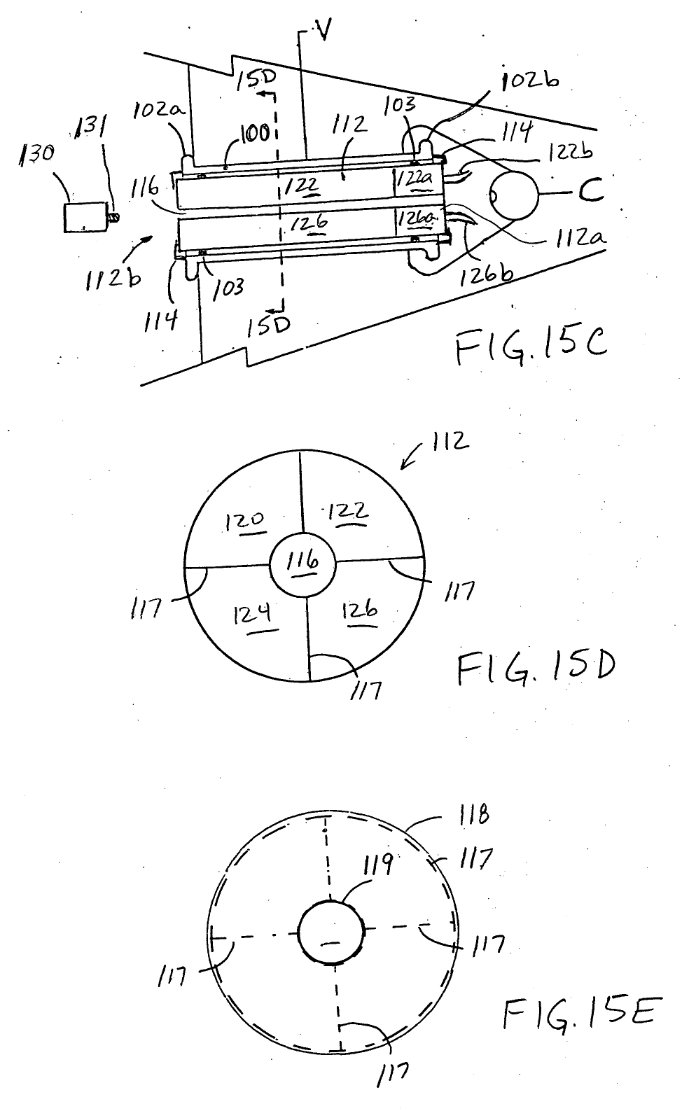

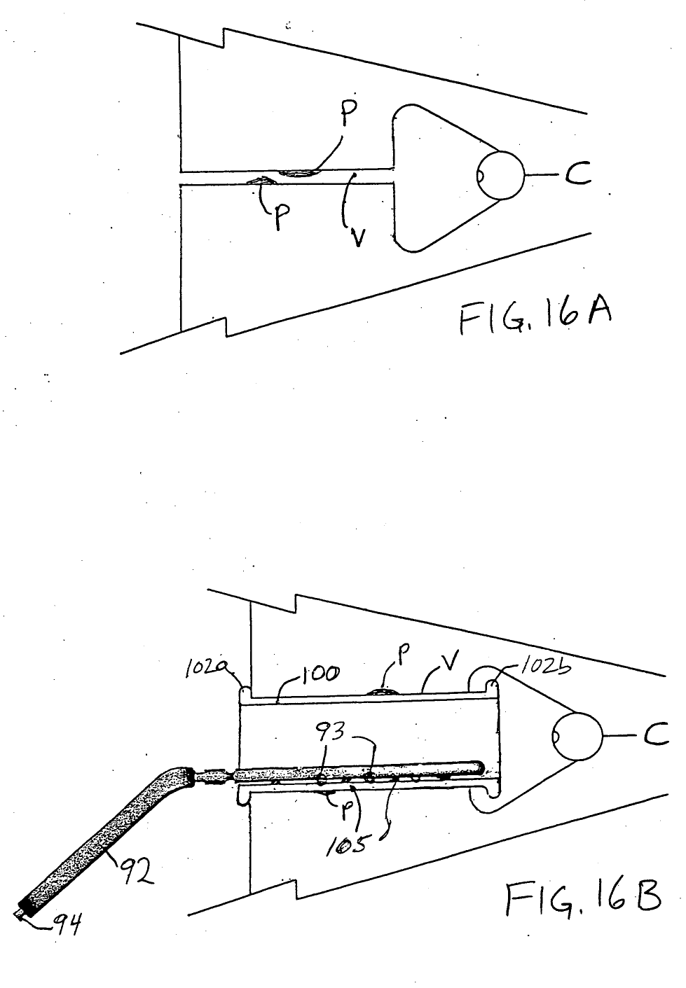

Referring to FIG. 15, handle assembly 90 and inflatable body 100, as disclosed with respect to FIGS. 12-13, are employed to facilitate delivery of drugs within a patient's vagina V. Handle assembly 90 is a modular assembly including intravaginal tongue portion 91 and detachable gripping portion 92. As described hereinabove with respect to FIG. 13, deflated inflatable body 100 and tongue portion 91 are inserted into vagina V and the inflatable body is inflated.

Referring to FIG. 15A, according to one aspect of the present invention, tongue portion 91 and inflatable body 100 are releasably connected via one or more fasteners 105. Fasteners 105 preferably comprise snaps or any other suitable interlocking releasable components or retaining members. During inflation, fasteners 105 maintain correct alignment between inflation holes 93 of tongue portion 91 and corresponding inflation holes in inflatable body 100. Referring to FIG. 13B, after inflation, fasteners 80 are unsnapped or otherwise released, thereby permitting tongue portion 91 to be removed entirely from the central lumen of the inflatable body.

Referring to FIG. 15C, after tongue portion 91 is retracted from the central lumen, cylinder 112 containing one or more drugs is positioned therein. More particularly, end 112a of cylinder 112 is inserted through cuff 102a, and cylinder is translated distally within the central lumen. Cylinder 112 preferably is locked into position within the central lumen by way of retaining tabs 114, or by other suitable fasteners. In the illustrated embodiment, distal and proximal ends 112a, 112b extend beyond the distal and proximal cuffs, respectively. Alternatively, distal and proximal ends 112a, 112b may be sized such that they are flush with distal and proximal cuffs 102b, 102a when disposed the within central lumen of the inflatable body.

Referring to FIG. 15D, cylinder 112 preferably comprises a plurality of distinct chambers 120, 122, 124, 126 disposed about lumen 116. Each chamber 120, 122, 124, 126 may be filled with one or more drugs adapted for continuous and/or intermittent delivery. Additionally, one or more of the chambers may be filled with other fluids such as water or saline. Chambers 120, 122, 124, 126 preferably include rigid or semi-rigid walls 117 that provide structural support to prevent the lateral vaginal wall from converging. Suitable combination materials for chamber walls 100 include silicon and polyurethane.

Referring to FIG. 15E, removable end cap 118 is provided to seal the proximal end of cylinder 112 during use. End cap 118 preferably is attached to cylinder 112 by way of force or friction fit. Additionally, end cap 118 may include aperture 119 aligned with lumen 116. Aperture 119 permits body fluids and excess drugs to be drained through lumen 116.

Referring again to FIG. 15C, each chamber 120, 122, 124, 126 preferably further contains a pump 120a, 122a, 124a, 126a in fluid communication with a corresponding nozzle 120b, 122b, 124b, 126b. Advantageously, the use of separate pumps prevents unnecessary mixing among drugs. Pumps 120a, 122a, 124a, 126a preferably include a timing mechanism for controlling the dispensing of predetermined quantities of drugs onto target treatment areas within vagina V. Nozzles 120b, 122b, 124b, 126b are preferably rotatable such that they may be pre-positioned, prior to insertion of cylinder 112, to dispense the drugs on a particular target treatment area. Although cylinder is depicted as having four cylinders, it will be appreciated by those of ordinary skill in the art that cylinder 112 may comprise any number of chambers (and corresponding pumps and nozzles) without departing from the scope of the present invention. Alternatively, cylinder 112 may comprise a plurality of chambers and a single pump/nozzle assembly that is configured to selectively dispense drugs from any one of the plurality of chambers.

With continued reference to FIG. 15C, one or more O-ring seals 103 are disposed between cylinder 112 and inflatable body 100 to prevent fluid leakage. According to some embodiments, lumen 100 is allowed to remain patent to permit natural drainage and to prevent infection. Cylinder preferably further comprises a handle 130 that has threaded end 131 for releasably engaging lumen 116 of cylinder 112. Handle 130 facilitates retraction of the cylinder from the central lumen of inflatable body 100. In addition, the handle may be used to rotate the cylinder to properly position it within the central lumen.

Drugs that have fast absorption properties are particularly suitable for delivery using cylinder 112 since the target tissue area where the drug is absorbed will be very close in proximity to the area where the drug is delivered. Drugs that may be delivered using cylinder 112 include, but are not limited to: antihypertensives, such as atenolol, diltiazem, enalapril, metoprolol and nifedipine; antibiotics; chronically administered drugs such as chemo; oral hypoglycemics such as glyburide, paclitaxol, abraxene and prep drug chremophor; drugs used to treat yeast infections such as terazol, diflucan, monistat and gynazole; and drugs used to treat bacterial infections such as flagy and cleocin.

According to one exemplary embodiment, chamber 120 is filled with chremophor and chamber 122 is filled with paclitaxol. The timing mechanism within pump 120a is configured to deliver a controlled amount of chremophor onto a target treatment area at specific time intervals, for example once every 3 days. Likewise, the timing mechanism within pump 122a is configured to deliver a controlled amount of paclitaxol onto the target treatment area at specific time intervals, for example once every 6 days. Alternatively, pumps 120a, 122b may be configured to continuously deliver chremophor and paclitaxol to the target treatment area.

According to another exemplary embodiment, designed to implement hormone replacement therapy in post-menopausal women or women who have had a hysterectomy, chamber 120 is filled with estrogen, chamber 122 is filled with progestin and chamber 124 is filled with water. The timing mechanism within pumps 120a, 122a are configured to deliver a controlled amount of estrogen and progestin, respectively, onto a target treatment area every week for three consecutive weeks. In the fourth week, the timing mechanism within pump 124a is configured to deliver a controlled amount of water to the target treatment area. This drug delivery schedule (i.e., three weeks of drugs followed by a week of water) preferably is repeated indefinitely until one of the chambers requires refilling by a physician.

The chambers in the cylinder are adapted to be filled periodically by a doctor or pharmacist. For example, a pharmacist may fill a prescription of multiple drugs into the chambers, wherein the prescription will last for a predetermined amount of time. Advantageously, being able to fill the chambers will multiple drugs provides a tremendous convenience for many patients, particularly older women. Additionally, the continuous administration of drugs by absorption provides less variability than taking oral doses.

Vaginal prolapse occurs when the vagina stretches such that its front or back wall bulges. In addition, prolapse can occur from the bladder, urethra, rectum, or uterus. Prolapse is managed in different ways depending upon the severity of the prolapse and the age of the patient. On one hand, younger patients commonly opt for a surgical solution, which is necessary because most younger patients are sexually active and a device in the vagina would be prohibitive. On the other hand, elderly patients are usually not sexually active. Therefore, the insertion of a device is typically preferred.

In general, insertable devices have not been very successful since they suffer from a number of drawbacks. As noted above, sexually active patients typically opt for surgery to avoid interruption of their sex life. Further, the devices are very uncomfortable to insert and remove and may cause infections. Thus, even for the elderly population, surgery is often the chosen solution. However, many of these patients are not excellent operative candidates. Prolapse frequently causes bulges or areas of prolapse in the vaginal wall. These areas of prolapse often exert a significant amount of pressure on the intra-vaginal device and tend to push conventional devices out of the vagina altogether.

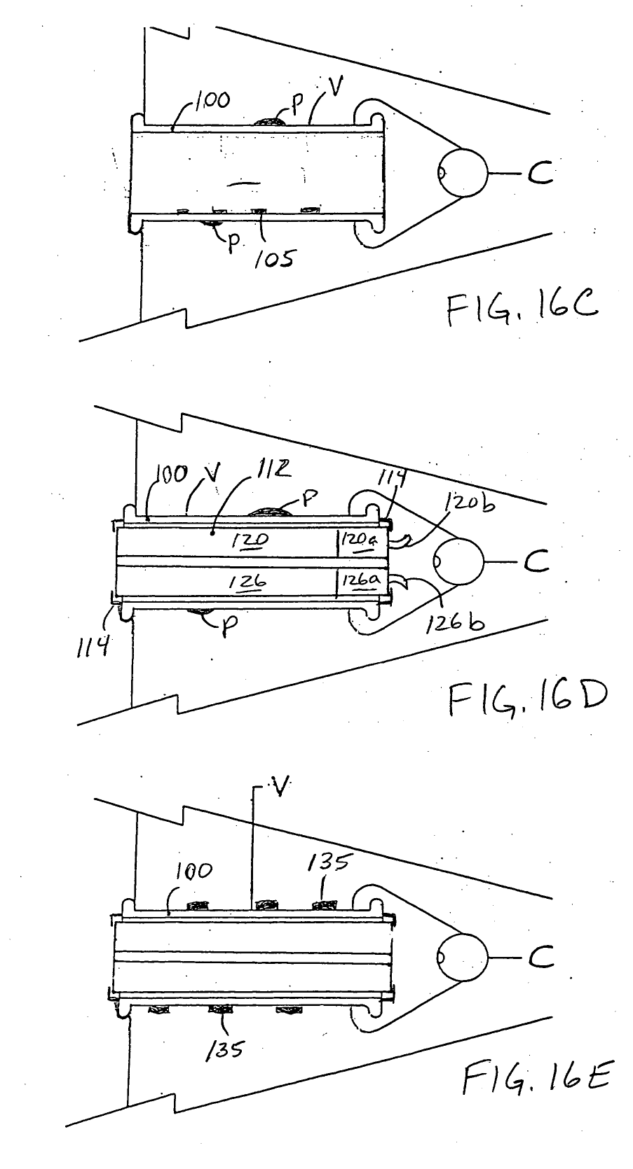

Referring to FIG. 16, handle assembly 90 and inflatable body 100, as disclosed with respect to FIGS. 12-13, are employed to relieve the symptoms of prolapse within a patient's vagina V. More particularly, the inflatable body is adapted to remain in the inflated configuration within vagina V for extended periods of time such as days, weeks or months. Referring to FIG. 16A, vagina V includes areas of prolapse P, which may cause incontinence, obstruction, discomfort and other symptoms. Referring to FIG. 16B, deflated inflatable body 100 and tongue portion 91 are inserted into vagina V and the inflatable body is inflated as described hereinabove. Tongue portion 91 and inflatable body 100 are releasably connected via one or more fasteners 105 so that tongue portion may be removed following inflation of inflatable body 100.

Referring to FIG. 16C, inflatable body 100 advantageously supports the vaginal cavity for extended periods of time, thereby preventing further areas of vaginal prolapse from developing. Further, the central lumen of inflatable body 100 provides a conduit for the passage of bodily fluids, thereby preventing obstruction due to the areas of prolapse. Moreover, inflatable body 100 is adapted to remain within the vaginal cavity for extended periods of time during which the areas of prolapse may recede or even disappear altogether.

According to some embodiments, the central lumen of the inflatable body remains patent such that it allows normal fluid to flow out. Advantageously, this inhibits infection and reduces odor from the build-up of old discharge. A waste reservoir may be provided to collect the fluid discharge. Referring to FIG. 16D, according to other embodiments, cylinder 112 such as described with respect to FIG. 15 may be inserted within the inflatable body. Cylinder 112 provides increased structural rigidity and a resulting increase in the lateral radial force of inflatable body 100. Of course, the increased lateral radial force helps keep the inflatable body within vagina V despite opposing forces from the areas of prolapse. Additionally, as depicted in FIG. 16E, the exterior wall of inflatable body 100 may include topographical features 135 that increase friction between the exterior wall and the vaginal wall. By way of example, topographical features 135 may include, but are not limited to, ribs, notches, ridges, indentations, bumps, grooves and other suitable surface irregularities.

Leep conization is a common procedure for women having pre-cancer of the cervix. The procedure employs a metal wire having a cauterizing current flowing through it, which is used to cut through the cervix and yield a specimen. In order to be usable for such procedures, conventional metal speculums must be manufactured with an electrically non-conductive coating, which makes such specula very expensive.

Referring to FIG. 17, inflatable body 100 is used to facilitate the excision of tissue from a patient's cervix C. In operation, deflated inflatable body 100 and tongue portion 91 are inserted into vagina V and the inflatable body is inflated as described hereinabove. Tongue portion 91 is then detached and retracted. Inflatable body 100 is non-metallic and therefore does not conduct electricity. Thus, the central lumen of the inflatable body provides a safe conduit with which to carry out a leep conization procedure.

Still referring to FIG. 17, the excision of tissue from cervix C is accomplished using metal wire 140, which has handle 141 attached at distal end 142 and electrode 143 attached to proximal end 144. Metal wire 140 and electrode 143 are in electrical communication with power source 148. Handle 141 is used to manipulate end 144 of metal wire 140 into endocervix E such that electrode 143 is in contact with cervix C. More particularly, the physician uses handle 141 to rotate and translate metal wire 140 with respect to inflatable body 100 and cervix C.

Once electrode 143 has been positioned in contact with cervix C, metal wire 140 is rotated using handle 141, thereby causing electrode 143 to excise a layer of cervical tissue. The metal wire then is retracted distally and the tissue sample is removed for processing. When the procedure is completed, the inflatable body is allowed to deflate automatically by the force exerted by the vaginal cavity.

As described hereinabove with respect to the embodiment of FIG. 5, an evacuation tube preferably is provided to evacuate smoke generated during excision of cervical tissue. For example, a flume may be created when the electrode contacts the cervix. Suction source 154 is provided to evacuate the flume through evacuation tube 152.

Intracavitary radiation devices exist for treatment of gynecologic cancers. One type of intracavitary radiation device comprises an oblong donut including a narrow central cylindrical opening. The radiation medium may be supplied as seeds and loaded into the device prior to placement of the device into the vagina. Such conventional intracavitary radiation devices must come in multiple sizes due to the large variation in vaginal cavity sizes. However, the conventional pre-sized devices do not often yield a comfortable fit for most patients.

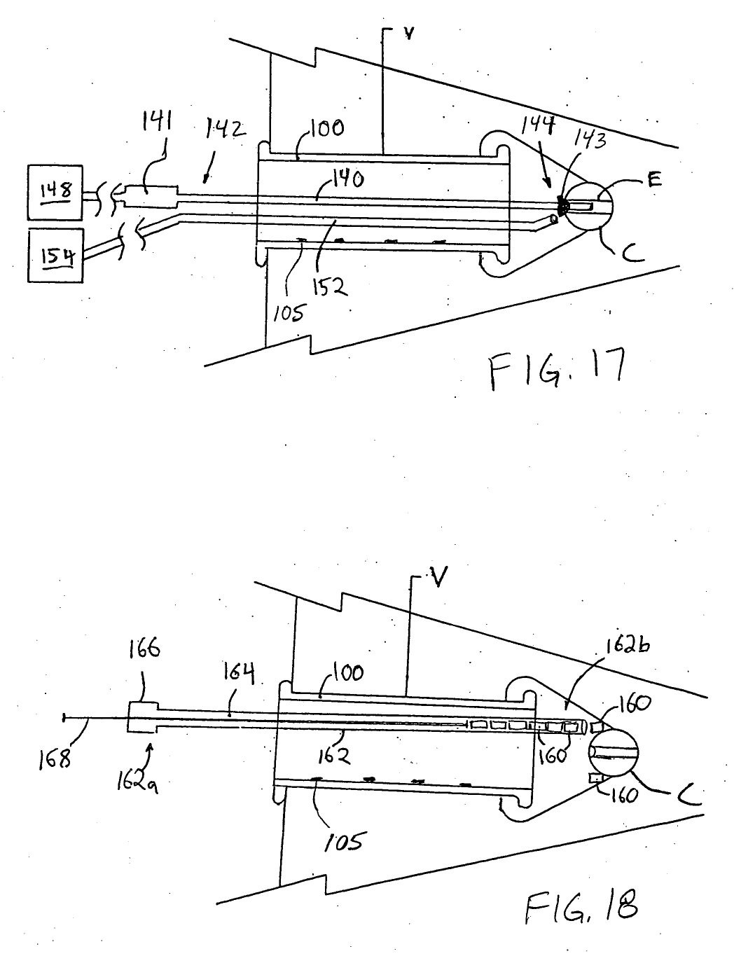

Referring to FIG. 18, inflatable body 100 of the present invention may be used to facilitate the delivery of therapeutic radiation to treat diseased tissue in the proximity of vagina V and cervix C. In operation, deflated inflatable body 100 and tongue portion 91 are inserted into vagina V and the inflatable body is inflated as described hereinabove. Tongue portion 91 is then detached and retracted from the central lumen of the inflatable body. According to one aspect of the present invention, the central lumen provides a conduit for delivery of radiation seeds 160 within vagina V. Radiation seeds 160 may be in the form of pellets, rods, tablets, globules, or any other suitable form.

Radiation seeds 160 are delivered to a diseased tissue area using elongated cylinder 162, which comprises distal end 162a and proximal end 162b and lumen 164 dimensioned for the passage of radiation seeds 160. Elongated cylinder 162 preferably further comprises a handle 166 disposed at distal end 162a. A physician uses handle 166 to manipulate proximal end 162b into position adjacent a diseased tissue area, for example the tissue surrounding cervix C. Once proximal end 162b has been properly positioned, push rod 168 is urged proximally within lumen 164 by the physician, thereby ejecting a radiation seed 160 from proximal end 162b. When the procedure is completed, the inflatable body is allowed to deflate automatically by the force exerted by the vaginal cavity. Advantageously, inflatable body 100 may be inflated to varying levels depending on the size of vagina V, thereby providing a comfortable fit for most patients.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

1. Apparatus for accessing a body cavity, comprising:
a tubular member having proximal and distal ends and an interior, the tubular member formed by everting a length of material upon itself to define a main body portion having a circumferential seam only at distal end, the tubular member having a deflated configuration for insertion into the body cavity and an expanded configuration when inflated; and
an inflation tube disposed in communication with an interior of the tubular member,
wherein the tubular member has a plurality of staggered contact areas arranged so that, in the expanded configuration, the contact areas do not form longitudinal features and the tubular member defines a substantially circular central lumen under load.

2. The apparatus of claim 1, wherein the tubular member further comprises proximal and distal cuffs.

3. The apparatus of claim 1, wherein the proximal end of the tubular member is configured to protect a patient's cervical area from rubbing or scraping.

4. The apparatus of claim 1, wherein the everted material forms an interior wall and an exterior wall and the inflation tube is interposed between the interior wall and the exterior wall.

5. The apparatus of claim 1, wherein the inflation tube is sufficiently rigid to transmit force when the tubular member is in the deflated configuration.

6. The apparatus of claim 1, further comprising a channel disposed within the central lumen, wherein the channel facilitates securing tools during a procedure.

7. The apparatus of claim 1, further comprising an inflator coupled to the inflation tube.

8. The apparatus of claim 7, further comprising a one-way valve interposed between the inflator and the tubular member.

9. The apparatus of claim 1, wherein the tubular member further comprises a lubricous exterior coating.

10. The apparatus of claim 1 further comprising a shield to reduce contamination of the inflator.

11. The apparatus of claim 1, wherein the tubular member further comprises a coating for topical application within the body cavity.

12. The apparatus of claim 11, wherein the coating contains drugs, gene vectors or proteins.

13. The apparatus of claim 11, wherein the coating includes a medication for treating yeast infections.

14. The apparatus of claim 1, wherein the tubular member further comprises an exterior layer including a plurality of micro-perforations for the passage of drugs in a liquid or gel form.

15. The apparatus of claim 1, further comprising a handle assembly including an intravaginal tongue portion and a gripping portion for holding and manipulating the tongue portion.

16. The apparatus of claim 15, wherein the tongue portion includes a lip configured to engage an end of a patient's vaginal canal.

17. The apparatus of claim 15, wherein the tongue portion is disposed at an angle relative to the gripping portion.

18. The apparatus of claim 15 wherein the handle assembly has a modular construction that allows the tongue portion to be detached from the gripping portion.

19. The apparatus of claim 18, wherein the tongue portion and gripping portion are releasably connected with a luer-type connector.

20. The apparatus of claim 1, further comprising a sheath that surrounds the inflatable body during insertion into the body cavity.

21. The apparatus of claim 1, further comprising a rigid support member that may be inserted within the tubular member to enhance the rigidity of the tubular member.

22. The apparatus of claim 1 further comprising a cylinder having one or more chambers, the cylinder configured to fit within the central lumen.

23. The apparatus of claim 15, wherein the handle assembly is releasably attached to the tubular member by one or more fasteners.

24. The apparatus of claim 22, wherein the one or more chambers are filled with bioactive substances.

25. The apparatus of claim 24, further comprising one or more pumps for dispensing the bioactive substances from the one or more chambers.

26. The apparatus of claim 22, wherein one or more chambers contain antihypertensives.

27. The apparatus of claim 22, wherein one or more chambers contain antibiotics.

28. The apparatus of claim 22, wherein one or more chambers contain a chronically administered drug.

29. The apparatus of claim 22, wherein one or more chambers contain oral hypoglycemics.

30. The apparatus of claim 22, wherein one or more chambers contain a drug used to treat yeast infections.

31. The apparatus of claim 22, wherein one or more chambers contain a drug used to treat bacterial infections.

32. The apparatus of claim 1 further comprising means for delivering radiation seeds within the body cavity.
